(19) United States
(12) Patent Application Publication
(10) Pub. No.: US 2026/0158082 A1
(43) Pub. Date: Jun. 11, 2026

(54) ALGORIPHAGUS SP, BOSEA SP, BREVUNDIMONAS SP, DESULFOVIBRIO SP, MICROBACTERIUM SP, SPHINGOMONAS SP, AND VARIOVORAX SP FOR USE IN DISEASE PREVENTION AND TREATMENT

(71) Applicant: ZIVO Bioscience, Inc., Troy, MI (US)

(72) Inventors: Amy E. Steffek, Royal Oak, MI (US); William P. Pfund, Kalamazoo, MI (US); Andrew A. Dahl, Bloomfield Hills, MI (US)

(73) Assignee: ZIVO Bioscience, Inc., Troy, MI (US)

(21) Appl. No.: 19/454,280

(22) Filed: Jan. 20, 2026

Related U.S. Application Data
(63) Continuation of application No. 17/576,339, filed on Jan. 14, 2022.
(60) Provisional application No. 63/138,041, filed on Jan. 15, 2021.

(51) Int. Cl. A61K 35/74; A23K 10/16; A23K 10/30; A61P 33/02

(52) U.S. Cl. CPC A61K 35/74; A23K 10/16; A23K 10/30; A61P 33/02

(57) ABSTRACT

A treatment compound and its use in the prevention and treatment diseases are disclosed. The compound comprises one or more materials selected from an algal biomass/supernatant (including both algae and bacteria), a bacterial biomass, and isolated and purified compound(s) as well as specific active sites or structures on those compounds. The treatment compound and the use of such a compound such as that derived from a bacterium that appears to selectively alter one or more TLR pathways in the prevention and treatment of disease in both animals and humans. The bacteria are selected from the group consisting of Algoriphagus sp., Bosea sp., Brevundimonas sp., Desulfovibrio sp., Microbacterium sp., Sphingomonas sp., and Variovorax sp. The disclosed inventive compound and method of treatment find particular application in the treatment of disease in poultry, particularly in the prevention and treatment of coccidiosis.

### TECHNICAL FIELD

The present disclosed inventive concept relates to the use of a novel compound in the treatment of various diseases in animals and humans by selectively altering TLR signaling pathways. More particularly, the disclosed inventive concept relates to a method and treatment using a treatment compound comprising one or more materials selected from an algal biomass/supernatant (including both algae and bacteria), a bacterial biomass, and isolated and purified compound(s) as well as specific active sites or structures on those compounds. Animals and humans treated with the inventive treatment compound experience reduced severity or entire elimination of certain diseases when compared with non-treated animal or human subjects. The treatment compound may be delivered orally as part of a natural feed composition or in the form of a capsule or tablet. The treatment compound may also be administered intravenously. The disclosed inventive concept has particular application in the poultry industry but may also find applications beyond poultry to other animals. The disclosed inventive concept may also be beneficial to humans.

### BACKGROUND OF THE INVENTION

The commercial animal industry is under constant economic pressure to develop methods of raising animals that maximize the number of commercially valuable members of a flock or herd. One such industry is the poultry industry which is facing dramatic increases in demand. Poultry meat competes with pork as the world's most consumed meat. It is expected that world poultry production will need to meet an increase in demand of over 120% by the year 2050.

Substantial economic losses in the poultry industry are most often the result of disease. Diseases in flocks often result in reduced production volume or compromised quality of meat. Prevention and treatment of poultry disease adds significantly to poultry production costs. Some estimates place total losses as a result of poultry disease at more than 10% of all production costs.

Of the diseases known to strike poultry flocks, the most common are enteric diseases which include coccidiosis, a disease caused by a parasite, the coccidian protozoa. Annual economic losses due to coccidiosis alone are estimated to exceed $3 billion per year and these costs are expected to increase due to a variety of reasons.

First, coccidiosis prevention today is accomplished mainly through the use of vaccines. A one-time administration of the vaccine is given very early in broiler life and, specifically, on the day of hatch. While this approach has shown some benefit, vaccines are known to suffer from variable effectiveness in controlling the disease over time. Experimentation has shown that a vaccine used in conjunction with a supplement such as a probiotic may improve outcome, but this approach faces its own challenges.

Second, coccidiosis treatment today is accomplished conventionally through the use of antibiotics and ionophores, both of which are costly. The use of antibiotics and ionophores is under pressure globally for a number of reasons, including environmental concerns related to the emergence of antibiotic-resistant pathogens. Drug resistance to antibiotics, ionophores, and synthetic treatment compounds is increasing largely due to overuse thereby severely compromising the effectiveness of these treatments. Relatively recently the European Union banned sub-therapeutic doses of certain antibiotics for use as feed additives. There has been no approval of new drugs in any of these categories for many years. Synthetic treatment compounds and other chemical agents are known but are not as effective as conventional antibiotics.

Third, even if known treatments were still economical and effective, known approaches would still be regarded as unsatisfactory because the medication must be included in the animal's feed for the full duration of its lifespan to be fully effective. This requirement adds significant cost to feed for the entire growout period.

Accordingly, it is desirable to develop a nonantibiotic-based treatment of pathogenic infections such as coccidiosis in poultry that is both practical and cost-effective.

### SUMMARY OF THE INVENTION

The disclosed inventive compound and method of treatment relates to a bacteria-based, compound for use in the prevention and treatment of a wide variety of diseases, including coccidiosis in poultry. More particularly, the present invention relates to a compound and the use of such a compound such as that derived from a bacterium that selectively alters one or more TLR pathways in the prevention and treatment of disease in both animals and humans. The bacteria are selected from the group consisting of Algoriphagus sp., Bosea sp., Brevundimonas sp., Desulfovibrio sp., Microbacterium sp., Sphingomonas sp., and Variovorax sp.

The compound of the disclosed inventive concept is combined with conventional feed for administration to animals such as poultry for the treatment of disease. The use of the inventive compound disclosed herein may also be used for the treatment of various diseases in humans. The combination of the disclosed inventive compound and conventional feed treats disease conditions by altering one or more TLR pathways. The disclosed inventive compound is a natural product and thus has no adverse environmental impact.

During the treatment period, the disclosed inventive compound is administered to the animal by way of poultry feed, in tablet or capsule form, in drinking water, or both along with a corn-soy based diet. The treatment compound may also be delivered intravenously. Studies based on the use of animal feed stock including specific variations of the disclosed inventive compound revealed improved health and disease prevention in animals. Data indicate that feeding chickens (specifically, broiler chickens) a corn/soy diet supplemented with a biomass comprising the inventive compound improves resistance to disease in healthy animals while providing improved treatment in diseased animals. It should be understood that while reference herein is made to a conventional diet of corn and soy, the disclosed compound may also be used to advantage in combination with other forms of conventional animal feed, such as, but not limited to, wheat. Evidence supports the conclusion that the inventive compound alters modulation of the various TLR pathways.

The disclosed inventive concept has numerous advantageous applications in humans and animals including but not limited to: (1) preventing disease in animals, particularly coccidiosis in poultry, (2) providing treatment to diseased animals, particularly in poultry suffering from coccidiosis, and (3) providing both disease prevention and disease treatment in an all-natural compound.

### DESCRIPTION OF THE DRAWINGS

For a more complete understanding of this invention, reference should now be made to the accompanying figures. As set forth in the figures, the designation “No Tx, No Challenge” refers to a test in which no treatment was administered to a subject animal not deliberately infected with coccidiosis. The designation “No Tx, Cocci” refers to a test in which no treatment was administered to a subject animal deliberately infected with coccidiosis. The designation “Anti-cocci, Cocci” refers to a test in which the subject animal was infected with coccidiosis and the animal was administered an anticoccidial.

The designation “ZIVO VP-UG” refers to a first test in which the subject animal was fed a composition having an algal biomass-derived portion which included V. paradoxus. The designation “ZIVO VP-BS” refers to a second test in which the subject animal was fed a composition having an algal biomass-derived portion which included V. paradoxus. The designation “ZIVO Sym-VP” refers to a test in which the subject animal was fed a composition having an algal biomass-derived portion which included Variovorax sp., Brevundimonas sp., Sphingomonas sp., and Microbacterium sp. The designation “ZIVO Sym” refers to a test in which the subject animal was fed a composition having an algal biomass-derived portion which included Brevundimonas sp., Sphingomonas sp., and Microbacterium sp.

The accompanying figures are described as follows:

FIG. 1 is a graph illustrating test subject feed conversion data for Days 1 to 28;

FIG. 2 is a graph illustrating test subject feed conversion data for Days 1 to 21.

FIG. 3 is a graph illustrating test subject feed conversion data for Days 1 to 14;

FIG. 4 is a graph illustrating test subject feed conversion data for Days 15 to 21;

FIG. 5 is a graph illustrating test subject feed conversion data for Days 22 to 28;

FIG. 6 is a graph illustrating test subject mortality for Days 1 to 28;

FIG. 7 is a graph illustrating test subject mortality for Days 1 to 21;

FIG. 8 is a graph illustrating test subject mortality for Days 1 to 14;

FIG. 9 is a graph illustrating test subject mortality for Days 15 to 21;

FIG. 10 is a graph illustrating test subject mortality for Days 22 to 28;

FIG. 11 is a graph illustrating test subject lesion scores determined on Day 14;

FIG. 12 is a graph illustrating test subject lesion scores determined on Day 28;

FIG. 13 is a graph illustrating test subject lesion score of the ceca determined on Day 14;

FIG. 14 is a graph illustrating test subject lesion score of the ceca determined on Day 28;

FIG. 15 is a graph illustrating test subject average body weight in grams on Days 1-14;

FIG. 16 is a graph illustrating test subject average weight gain in grams per day on Days 1-21;

FIG. 17 is a graph illustrating test subject average body weight in grams on Days 1-28;

FIG. 18 is a graph illustrating test subject average body weight in grams on Days 1-14 (additional test);

FIG. 19 is a graph illustrating test subject average body weight in grams on Days 15-21 (additional test);

FIG. 20 is a graph illustrating test subject average body weight in grams on Days 22-28 (additional test);

FIG. 21 is a graph illustrating test subject feed intake on Days 1-14;

FIG. 22 is a graph illustrating test subject feed intake on Days 1-21;

FIG. 23 is a graph illustrating test subject feed intake on Days 1-28;

FIG. 24 is a graph illustrating test subject feed intake on Days 15 to 21; and

FIG. 25 is a graph illustrating test subject feed intake on Days 22-28.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The method of the disclosed inventive concept proposes the use of a compound in the prevention and treatment of disease in both animals and humans. The treatment compound comprises one or more materials selected from an algal biomass/supernatant (including both algae and bacteria), symbiont bacteria, bacterial biomass, bacterial fermentate, and isolated and purified compound(s) as well as specific active sites or structures on those compounds. The inventive compound is combined with conventional feed to create a feed mixture that is fed to chickens, for example, broiler chickens, as well as other animals, to both prevent disease in healthy animals and to treat disease in diseased animals.

### The Compound Used in Growth Promotion Method and Treatment

The disclosed growth promotion method and treatment utilizes an effective disease prevention and treatment compound comprising one or more materials selected from an algal biomass/supernatant (including both algae and bacteria), a bacterial biomass, and isolated and purified compound(s) as well as specific active sites or structures on those compounds. By administering the compound early in broiler life, disease may be prevented. By administering the compound to a diseased animal, treatment can be achieved. The effective compound may be derived from the lipopolysaccharide (LPS) layer of a gram-negative bacteria or may be derived from a source other than a lipopolysaccharide.

As used herein, the terms “alteration” or “alter” relate to the impact of a molecule that alters the activity induced by another molecule. By way of example, a compound that might block the LPS-dependent modulation of TLR receptors (including, but not limited to TLR2, TLR3, TLR4, TLR6, TLR7, TLR8, and/or TLR9 receptors) present on the surface of immune cells in humans and animals would be regarded as altering this particular pathway.

As used herein, the term “bacterial culture” is defined as a bacterial organism (one or more types) that grow alone or together in a liquid medium. Unless expressly stated otherwise, the term “bacterial biomass” refers to the bacterial cells (with the liquid culture medium removed). The “bacterial biomass” can be wet material or dried material.

Unless expressly stated otherwise, the term “bacterial supernatant” is defined as the culture medium in which the bacterial biomass is grown that contains excreted compounds from the bacterial biomass. Bacterial supernatant is obtained by growing bacterial biomass in culture medium for an appropriate length of time and then removing the bacterial cells by filtration and/or centrifugation.

Embodiments of the compound used in the growth promotion method and treatment as set forth herein include one or more LPS/Lipid A compounds produced by gram-negative bacterial strains for use in the alteration of one or more of the TLR signaling pathways. The bacterial strains include one or more of the following: Algoriphaqus sp., Bosea sp., Brevundimonas sp., Desulfovibrio sp., Microbacterium sp., Sphingomonas sp., and Variovorax sp. Specific species of these bacteria may include one or more of the following: Algoriphaqus aquaticus, Algoriphagus aquatilis, Bosea nasdae, Brevundimonas diminuta, Brevundimonas vesicularis, Microbacterium testaceum, and Variovorax paradoxus.

Algoriphagus is a genus of Gram-negative, non-spore-forming, non-motile bacterium found in the biofilm of a freshwater lake.

Bosea is a genus of bacteria from the family of Bradyrhizobiaceae having ten genera and include plant-associated bacteria such as Bradyrhizobium, a genus of rhizobia associated with some legumes.

Brevundimonas is a genus of Proteobacteria, Gram-negative, non-fermenting, aerobic bacilli. The Brevundimonas species are ubiquitous in the environment.

Desulfovibrio is a genus of Gram-negative, sulfate-reducing bacteria and are commonly found in aquatic environments with high levels of organic material, as well as in water-logged soils.

Microbacterium is a genus of Gram-positive endophytic bacterium that resides within plant hosts without causing disease symptoms.

Sphingomonas is a genus of Gram-negative, rod-shaped, chemoheterotrophic, strictly aerobic bacteria.

Variovorax is a genus of Gram-negative aerobic bacterium that can grow under a variety of conditions. It is part of the subclass Proteobacteria and is capable of metabolically utilizing several natural compounds generated by plants or algae.

The specific bacteria investigated in the present application include species of Bosea, Microbacterium, Sphingomonas, and Variovorax. However, it is to be understood that the other bacteria listed above may prove equally effective in the prevention and treatment of disease.

The disclosed inventive concept involves any combination of two fundamental steps: (1) the gram-negative bacteria produce LPS/Lipid A compounds and (2) the LPS/Lipid compounds modulate TLR activity by altering the signaling pathway thereby preventing or reversing diseases such as coccidiosis. In an embodiment, the LPS/Lipid A compounds produced by the above-noted bacteria used to selectively alter the TLR signaling pathway (including, but not limited to TLR2, TLR3, TLR4, TLR6, TLR7, TLR8, and/or TLR9 receptors). The strains may be naturally occurring and may be found in a variety of environments and natural materials.

The LPS/Lipid A compound employed herein may be obtained from the bacterial strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1% deoxycholate and, optionally; (5) performing additional purification using size-exclusion chromatography.

### Disease Prevention and Treatment-Data

### General Study Information

Non-limiting examples of a composition and method for preventing and treating disease in animals and humans are set forth. It is to be understood that while the following method is directed to the enhancement of growth in poultry, the disclosed method may apply as well to other animals as well as humans. Accordingly, the described growth promotion method and treatment is not intended as being solely for use in poultry.

According to the present, non-limiting examples, the inventive compound is defined as the bacterial biomass as set forth above and related materials including bacterial supernatant. The inventive compound was mixed with conventional feed to form a supplemented “feed mixture” at a fixed ratio. This ratio was maintained throughout the test period. The bird flock was divided into a control group fed only conventional corn-soy feed and an experimental group fed the supplemented feed mixture.

A study was undertaken to determine the response and efficacy of a dried algal biomass feed ingredient incorporated at a specific amount into a commercial-type corn-soybean diet and fed to floor-pen raised broilers. The study was undertaken over a 28-day period, from Day 0 to Day 28. Particularly, the treatment compound is fresh water algal biomass containing Gram-negative bacteria provided as animal feed in combination of a feed additive, such as soy oil, preferably though not exclusively at a ratio of two parts soil oil to one part algal biomass. Once the biomass and feed additive are combined to the preferred premix level, the combined batch is poured or administered evenly into a ribbon mixer containing finished feed.

Two non-limiting embodiments of the biomass provided as animal feed in combination with feed additive are disclosed. The biomass of the first embodiment included a cocktail blend of four bacteria while the biomass of the second embodiment included a blend of three bacteria. A greater or lesser number of bacteria may be included as part of the blend.

With respect to the first embodiment, a blend of four bacteria from the genera Variovorax, Sphingomonas, Brevundimonas, and Microbacterium are provided in the biomass in a total amount preferably of between about 100.0 g per ton of finished feed and about 150.0 g per ton of finished feed, more preferably provided in an amount of between about 120.0 g per ton of finished feed and 130.0 g per ton of finished fee, and is most preferably though not exclusively in an amount of about 126.0 g per ton of feed with good efficacy without being wasteful.

The quantity of the bacteria forming the blend may be varied. As non-limiting examples, both Variovorax and Brevundimonas preferably represent between about 30.0 g and 60.0 g of the total biomass, more preferably between about 40.0 g and 50.0 g of the total biomass, and most preferably about 45.0 g of the biomass, while Sphingomonas preferably represents between about 15.0 g and 45.0 g of the total biomass, more preferably between about 25.0 g and 35.0 g of the total biomass, and most preferably about 30.0 g of the biomass and Microbacterium preferably represents between about 0.1 g and 20.0 g of the total biomass, more preferably between about 1.0 g and 10.0 g of the total biomass, and most preferably about 6.0 g of the biomass.

With respect to the second embodiment, a blend of three bacteria from the genera Sphingomonas, Brevundimonas, and Microbacterium are provided in the biomass in a total amount preferably of between about 60.0 g per ton of finished feed and about 100.0 g per ton of finished feed, more preferably provided in an amount of between about 70.0 g per ton of finished feed and 90.0 g per ton of finished fee, and is most preferably though not exclusively in an amount of about 81.0 g per ton of feed with good efficacy without being wasteful.

The quantity of the bacteria forming the blend may be varied. As non-limiting examples, Brevundimonas preferably represents between about 30.0 g and 60.0 g of the total biomass, more preferably between about 40.0 g and 50.0 g of the total biomass, and most preferably about 45.0 g of the biomass, while Sphingomonas preferably represents between about 15.0 g and 45.0 g of the total biomass, more preferably between about 25.0 g and 35.0 g of the total biomass, and most preferably about 30.0 g of the biomass and Microbacterium preferably represents between about 0.1 g and 20.0 g of the total biomass, more preferably between about 1.0 g and 10.0 g of the total biomass, and most preferably about 6.0 g of the biomass.

### Study—Treatment Method

A total of 1,680 mixed sex broiler chicks were obtained from a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study. Bird replicates were 20 with 12 replicates per treatment groups. There were seven treatment groups.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets which included algal fermentate of the present composition as pellets and were fed their respective treatment diets ad libitum from day of hatch to 28 days of age. Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds were challenged by receiving an oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

### Cocci-Challenge—

On Day 7, all birds received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

### Study—Groupings

Seven study groups were established as set forth below.

| | |
|---|---|
| Group 1 (control) | No Challenge, No Tx |
| Group 2 (control) | Cocci Challenge, No Tx |
| Group 3 (control) | Cocci Challenge, Anti-cocci Tx (Coban ®) |
| Group 4 | 45 g/ton UGA¹ V. paradoxus Biomass |
| | ¹First Research Institute |
| Group 5 | 45 g/ton BioSource² V. paradoxus Biomass |
| | ²Second Research Institute |
| Group 6 | 126 g/ton Symbiont Cocktail w/V. paradoxus |
| | 45 g (0.36 ratio) UGA V. paradoxus Biomass |
| | 30 g (0.24 ratio) BioSource² Sphingomonas Biomass |
| | 45 g (0.36 ratio) UGA B. nasdae Biomass |
| | 6 g (0.05 ratio) BioSource Microbacteriaum sp. |
| Group 7 | 81 g/ton Symbiont Cocktail w/o V. paradoxus |
| | 30 g (0.37 ratio) BioSource Sphingomonas Biomass |
| | 45 g (0.56 ratio) UGA B. nasdae Biomass |
| | 6 g (0.07 ratio) BioSource Microbacterium sp |

### Study—Results

As used below, “FCR” refers to “feed conversion ratio.” FCR conventionally represents a ratio (or a rate) of measuring efficiency in animals in converting feed to a specific output.

As used below, “VP-UG” refers to the treatment compound “UGA V. paradoxus Biomass” previously noted.

As used below, “VP-BS” refers to the treatment compound “BioSource V. paradoxus Biomass” previously noted.

As used below, “Sym-VP” refers to the “Symbiont Cocktail w/V. paradoxus” previously noted.

As used below, “Sym” refers to the “Symbiont Cocktail w/o V. paradoxus” previously noted.

The data below are based on 125 g/ton of the Symbiont Cocktail with V. paradoxus as opposed to 126 g/ton of the same cocktail blend and on 80 g/ton of the Symbiont Cocktail without V. paradoxus as opposed to 81 g/ton of the same cocktail as identified above. However, the ratios of the individual components remained the same.

### Data—Tables

The following tables, when taken in conjunction with the accompanying figures, provide support for the effectiveness of the composition and treatment method of the present invention as disclosed herein.

TABLE No. 1

| | FCR | FCR | FCR | | | |
|---|---|---|---|---|---|---|
| | Corrected | Corrected | Corrected | Mortality | Mortality | Mortality |
| Mean | D1-14 | D1-21 | D1-28 | D1-14 | D1-21 | D1-28 |
| 1. No Tx, No Challenge | 1.090 | 1.155 | 1.385 | 0.417 | 1.042 | 1.042 |
| 2. No Tx, Cocci | 1.142 | 1.217 | 1.434 | 3.333 | 7.813 | 8.854 |
| 3. Anti-cocci, Cocci* | 1.112 | 1.151 | 1.387 | 0.833 | 1.563 | 1.563 |
| 4. ZIVO VP-UG (45 g/ton) | 1.126 | 1.184 | 1.412 | 1.667 | 4.167 | 5.208 |
| 5. ZIVO VP-BS (45 g/ton) | 1.126 | 1.181 | 1.419 | 1.667 | 4.167 | 5.729 |
| 6. ZIVO Sym-VP (125 g/ton) | 1.129 | 1.165 | 1.392 | 1.250 | 2.604 | 2.604 |
| 7. ZIVO Sym (80 g/ton) | 1.128 | 1.169 | 1.401 | 1.667 | 3.125 | 3.125 |
| Stats | STAT* | STAT* | STAT* | STAT* | STAT* | STAT* |
| 1. No Tx, No Challenge | a | ab | a | a | a | a |
| 2. No Tx, Cocci | c | c | b | b | b | d |
| 3. Anti-cocci, Cocci | ab | a | a | a | a | ab |
| 4. ZIVO VP-UG (45 g/ton) | bc | b | ab | ab | a | bcd |
| 5. ZIVO VP-BS (45 g/ton) | bc | ab | ab | ab | a | cd |
| 6. ZIVO Sym-VP (125 g/ton) | bc | ab | a | a | a | abc |
| 7. ZIVO Sym (80 g/ton) | bc | ab | ab | ab | a | abc |
| StdDev | S.D. | S.D. | S.D. | S.D. | S.D. | S.D. |
| 1. No Tx, No Challenge | 0.03 | 0.03 | 0.04 | 1.38 | 2.33 | 2.33 |
| 2. No Tx, Cocci | 0.03 | 0.03 | 0.04 | 2.36 | 4.51 | 5.96 |
| 3. Anti-cocci, Cocci | 0.03 | 0.04 | 0.04 | 1.86 | 2.71 | 2.71 |
| 4. ZIVO VP-UG (45 g/ton) | 0.02 | 0.03 | 0.04 | 2.36 | 3.90 | 5.61 |
| 5. ZIVO VP-BS (45 g/ton) | 0.03 | 0.04 | 0.03 | 2.36 | 3.90 | 4.75 |
| 6. ZIVO Sym-VP (125 g/ton) | 0.04 | 0.04 | 0.04 | 2.17 | 4.00 | 4.00 |
| 7. ZIVO Sym (80 g/ton) | 0.03 | 0.03 | 0.04 | 2.36 | 4.03 | 4.03 |

*Commercial Anti-Cocci Comparator

TABLE NO. 2

| | Mean | | | |
|---|---|---|---|---|
| | D 14 Avg | D 28 Avg | D 14 Avg | D 28 Avg |
| | Lesion Score | Lesion Score | Lesion Score | Lesion Score |
| | Duodenum | Duodenum | ceca | ceca |
| 1. No Tx, No Challenge | 0.125 | 0.167 | 0.146 | 0.188 |
| 2. No Tx, Cocci | 1.583 | 1.563 | 1.729 | 1.667 |
| 3. Anti-cocci, Cocci | 0.375 | 0.208 | 0.292 | 0.229 |
| 4. ZIVO VP-UG (45 g/ton) | 0.917 | 1.083 | 0.896 | 0.875 |
| 5. ZIVO VP-BS (45 g/ton) | 0.896 | 1.000 | 0.896 | 1.021 |
| 6. ZIVO Sym-VP (125 g/ton) | 0.646 | 0.625 | 0.667 | 0.563 |
| 7. ZIVO Sym (80 g/ton) | 0.604 | 0.583 | 0.646 | 0.667 |
| Stats | STAT* | STAT* | STAT* | STAT* |
| 1. No Tx, No Challenge | a | a | a | a |
| 2. No Tx, Cocci | e | d | d | d |
| 3. Anti-cocci, Cocci | b | a | a | a |
| 4. ZIVO VP-UG (45 g/ton) | d | c | c | c |
| 5. ZIVO VP-BS (45 g/ton) | d | c | c | c |
| 6. ZIVO Sym-VP (126 g/ton) | c | b | b | b |
| 7. ZIVO Sym (81 g/ton) | c | b | b | b |
| StdDev | S.D. | S.D. | S.D. | S.D. |
| 1. No Tx, No Challenge | 0.13 | 0.12 | 0.16 | 0.15 |
| 2. No Tx, Cocci | 0.16 | 0.21 | 0.22 | 0.19 |
| 3. Anti-cocci, Cocci | 0.22 | 0.20 | 0.22 | 0.26 |
| 4. ZIVO VP-UG (45 g/ton) | 0.26 | 0.24 | 0.26 | 0.28 |
| 5. ZIVO VP-BS (45 g/ton) | 0.28 | 0.31 | 0.26 | 0.36 |
| 6. ZIVO Sym-VP (126 g/ton) | 0.16 | 0.30 | 0.16 | 0.25 |
| 7. ZIVO Sym (81 g/ton) | 0.26 | 0.24 | 0.26 | 0.21 |

TABLE No. 3

| | | | | | D1-14 | D1-21 | D1-28 |
|---|---|---|---|---|---|---|---|
| | | | | | Feed | Feed | Feed |
| | Avg BW | Avg BW | Avg BW | Avg BW | Intake | Intake | Intake |
| Mean | D1 | D1-14 | D1-21 | D1-28 | (g/bird/day) | (g/bird/day) | (g/bird/day) |
| 1. No Tx, No Challenge | 54.663 | 481.68 | 914.50 | 1451.70 | 33.246 | 53.309 | 74.570 |
| 2. No Tx, Cocci | 54.563 | 450.52 | 835.70 | 1288.10 | 32.790 | 52.848 | 70.520 |
| 3. Anti-cocci, Cocci | 54.663 | 496.13 | 951.80 | 1535.00 | 34.680 | 54.811 | 78.960 |
| 4. ZIVO VP-UG (45 g/ton) | 54.563 | 467.82 | 877.90 | 1383.10 | 33.389 | 53.307 | 73.742 |
| 5. ZIVO VP-BS (45 g/ton) | 54.579 | 462.16 | 875.30 | 1383.70 | 32.991 | 52.968 | 74.313 |
| 6. ZIVO Sym-VP (125 g/ton) | 54.592 | 483.01 | 911.10 | 1466.80 | 34.713 | 54.198 | 76.171 |
| 7. ZIVO Sym (80 g/ton) | 54.683 | 483.68 | 914.50 | 1474.90 | 34.760 | 54.693 | 77.216 |
| Stats | STAT* | STAT* | STAT* | STAT* | STAT* | STAT* | STAT* |
| 1. No Tx, No Challenge | a | b | b | b | a | a | b |
| 2. No Tx, Cocci | a | d | d | d | a | a | a |
| 3. Anti-cocci, Cocci | a | a | a | a | b | a | c |
| 4. ZIVO VP-UG (45 g/ton) | a | c | c | c | a | a | ab |
| 5. ZIVO VP-BS (45 g/ton) | a | c | c | c | a | a | b |
| 6. ZIVO Sym-VP (125 g/ton) | a | b | b | b | b | a | bc |
| 7. ZIVO Sym (80 g/ton) | a | b | b | b | b | a | bc |
| StdDev | S.D. | S.D. | S.D. | S.D. | S.D. | S.D. | S.D. |
| 1. No Tx, No Challenge | 0.23 | 11.17 | 26.05 | 37.91 | 0.84 | 1.56 | 3.66 |
| 2. No Tx, Cocci | 0.30 | 16.89 | 31.31 | 44.10 | 1.60 | 2.58 | 3.72 |
| 3. Anti-cocci, Cocci | 0.33 | 11.83 | 28.91 | 53.50 | 1.51 | 2.78 | 3.44 |
| 4. ZIVO VP-UG (45 g/ton) | 0.28 | 15.35 | 38.87 | 39.55 | 1.56 | 2.88 | 4.54 |
| 5. ZIVO VP-BS (45 g/ton) | 0.25 | 12.30 | 32.59 | 60.38 | 1.16 | 3.22 | 4.46 |
| 6. ZIVO Sym-VP (125 g/ton) | 0.29 | 10.27 | 30.98 | 50.94 | 1.56 | 2.99 | 3.17 |
| 7. ZIVO Sym (80 g/ton) | 0.32 | 16.39 | 25.80 | 38.43 | 1.73 | 2.11 | 3.22 |

TABLE No. 4

| | FCR | FCR | FCR | | | |
|---|---|---|---|---|---|---|
| | Corrected | Corrected | Corrected | Mortality | Mortality | Mortality |
| Mean | D1-14 | D15-21 | D22-28 | D1-14 | D15-21 | D22-28 |
| 1. No Tx, No Challenge | 1.090 | 1.239 | 1.805 | 0.417 | 0.625 | 0.000 |
| 2. No Tx, Cocci | 1.142 | 1.323 | 1.888 | 3.333 | 4.479 | 1.042 |
| 3. Anti-cocci, Cocci | 1.112 | 1.244 | 1.825 | 0.833 | 0.729 | 0.000 |
| 4. ZIVO VP-UG (45 g/ton) | 1.126 | 1.262 | 1.851 | 1.667 | 2.500 | 1.042 |
| 5. ZIVO VP-BS (45 g/ton) | 1.126 | 1.251 | 1.875 | 1.667 | 2.500 | 1.563 |
| 6. ZIVO Sym-VP (125 g/ton) | 1.129 | 1.208 | 1.794 | 1.250 | 1.354 | 0.000 |
| 7. ZIVO Sym (80 g/ton) | 1.128 | 1.222 | 1.815 | 1.667 | 1.458 | 0.000 |
| Stats | STAT* | STAT* | STAT* | STAT* | STAT* | STAT* |
| 1. No Tx, No Challenge | a | ab | a | a | a | a |
| 2. No Tx, Cocci | c | b | a | b | b | ab |
| 3. Anti-cocci, Cocci | ab | ab | a | a | a | a |
| 4. ZIVO VP-UG (45 g/ton) | bc | ab | a | ab | ab | ab |
| 5. ZIVO VP-BS (45 g/ton) | bc | ab | a | ab | ab | c |
| 6. ZIVO Sym-VP (125 g/ton) | bc | a | a | a | a | a |
| 7. ZIVO Sym (80 g/ton) | bc | a | a | ab | a | a |
| StdDev | S.D. | S.D. | S.D. | S.D. | S.D. | S.D. |
| 1. No Tx, No Challenge | 0.03 | 0.1 | 0.11 | 1.38 | 1.73 | 0 |
| 2. No Tx, Cocci | 0.03 | 0.08 | 0.13 | 2.36 | 3.21 | 2.33 |
| 3. Anti-cocci, Cocci | 0.03 | 0.16 | 0.17 | 1.86 | 1.73 | 0 |
| 4. ZIVO VP-UG (45 g/ton) | 0.02 | 0.06 | 0.16 | 2.36 | 2.93 | 2.33 |
| 5. ZIVO VP-BS (45 g/ton) | 0.03 | 0.11 | 0.13 | 2.36 | 2.93 | 2.71 |
| 6. ZIVO Sym-VP (125 g/ton) | 0.04 | 0.12 | 0.12 | 2.17 | 2.52 | 0 |
| 7. ZIVO Sym (80 g/ton) | 0.03 | 0.06 | 0.14 | 2.36 | 2.49 | 0 |

TABLE No. 5

| | Avg BW | Avg BW | Avg BW | Feed Intake | Feed Intake | Feed Intake |
|---|---|---|---|---|---|---|
| | gain | gain | gain | (g/bird/day) | (g/bird/day) | (g/bird/day) |
| Mean | D1-14 | D15-21 | D22-28 | D1-14 | D15-21 | D22-28 |
| 1. No Tx, No Challenge | 427.00 | 432.8 | 537.2 | 33.246 | 76.236 | 138.354 |
| 2. No Tx, Cocci | 396.00 | 385.2 | 452.5 | 32.790 | 72.483 | 121.443 |
| 3. Anti-cocci, Cocci | 441.50 | 445.2 | 583.2 | 34.680 | 77.989 | 151.408 |
| 4. ZIVO VP-UG (45 g/ton) | 413.30 | 410.1 | 505.2 | 33.389 | 74.179 | 132.894 |
| 5. ZIVO VP-BS (45 g/ton) | 407.60 | 413.1 | 508.4 | 32.991 | 74.146 | 135.576 |
| 6. ZIVO Sym-VP (125 g/ton) | 428.40 | 428.1 | 555.7 | 34.713 | 74.493 | 142.091 |
| 7. ZIVO Sym (80 g/ton) | 429.00 | 430.8 | 560.4 | 34.760 | 75.735 | 144.785 |
| Stats | STAT* | STAT* | STAT* | STAT* | STAT* | STAT* |
| 1. No Tx, No Challenge | b | a | abc | a | a | ab |
| 2. No Tx, Cocci | d | b | d | a | a | c |
| 3. Anti-cocci, Cocci | a | a | a | b | a | a |
| 4. ZIVO VP-UG (45 g/ton) | c | ab | c | a | a | bc |
| 5. ZIVO VP-BS (45 g/ton) | c | ab | bc | a | a | b |
| 6. ZIVO Sym-VP (125 g/ton) | b | a | abc | b | a | ab |
| 7. ZIVO Sym (80 g/ton) | b | a | ab | b | a | ab |
| StdDev | S.D. | S.D. | S.D. | S.D. | S.D. | S.D. |
| 1. No Tx, No Challenge | 11.17 | 26.70 | 50.60 | 0.84 | 5.10 | 13.85 |
| 2. No Tx, Cocci | 16.87 | 38.76 | 60.10 | 1.60 | 6.53 | 14.31 |
| 3. Anti-cocci, Cocci | 11.82 | 44.73 | 59.68 | 1.51 | 8.08 | 14.81 |
| 4. ZIVO VP-UG (45 g/ton) | 15.33 | 44.41 | 54.32 | 1.56 | 8.22 | 13.49 |
| 5. ZIVO VP-BS (45 g/ton) | 12.20 | 35.06 | 70.85 | 1.16 | 8.73 | 18.19 |
| 6. ZIVO Sym-VP (125 g/ton) | 10.18 | 34.24 | 43.35 | 1.56 | 10.72 | 10.46 |
| 7. ZIVO Sym (80 g/ton) | 16.35 | 23.50 | 58.47 | 1.73 | 5.23 | 14.99 |

### Results

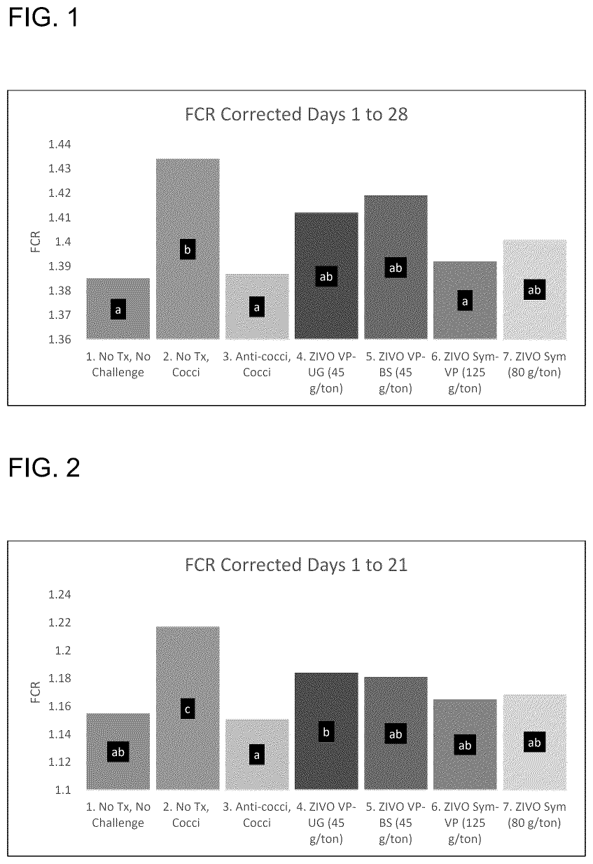

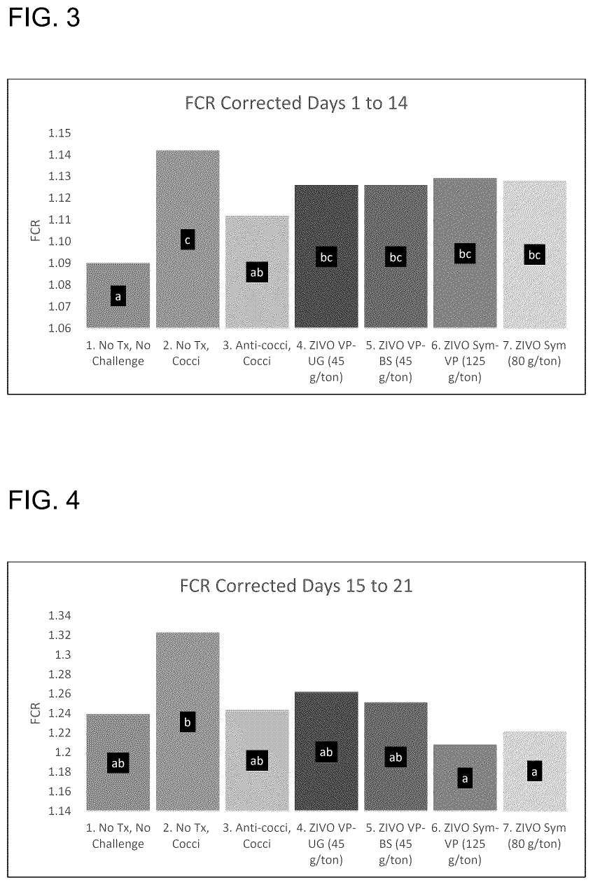

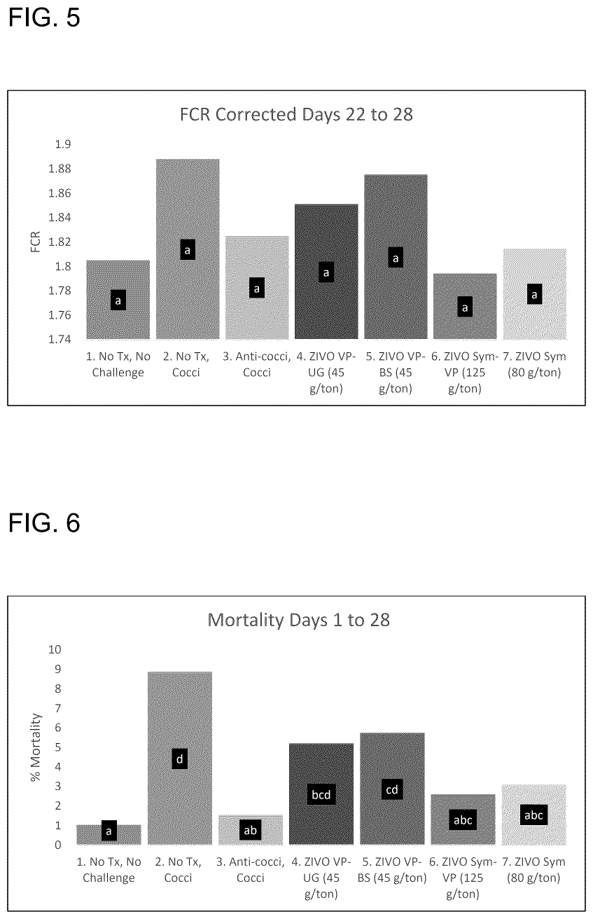

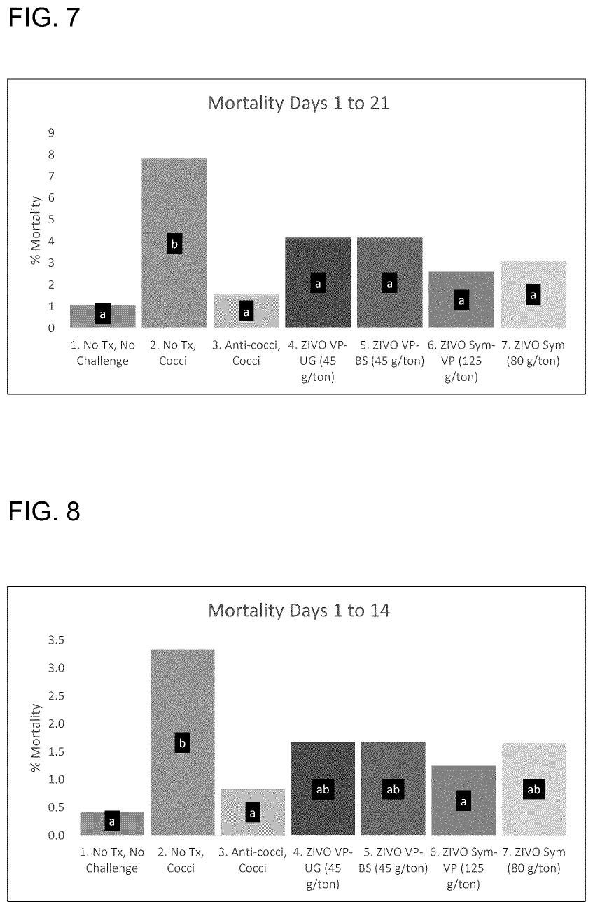

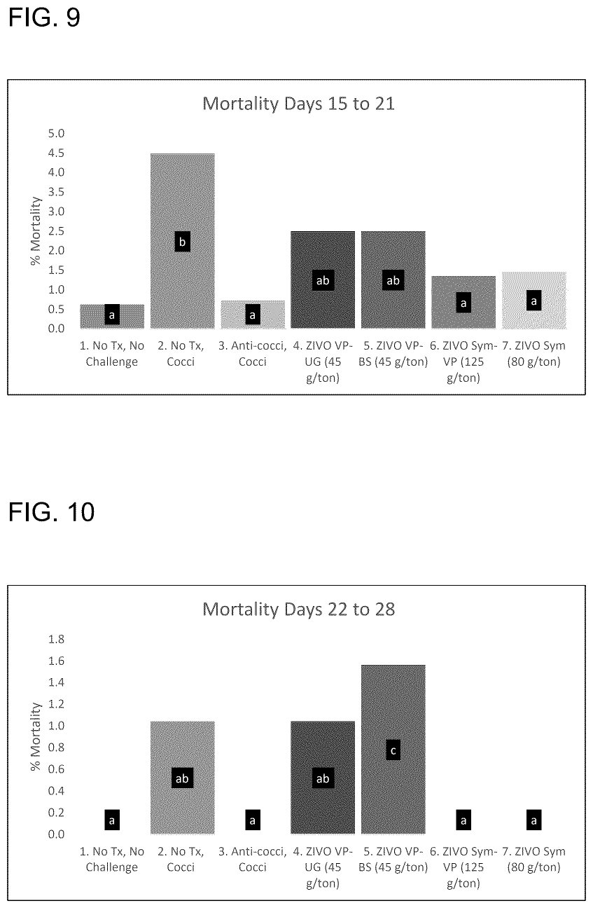

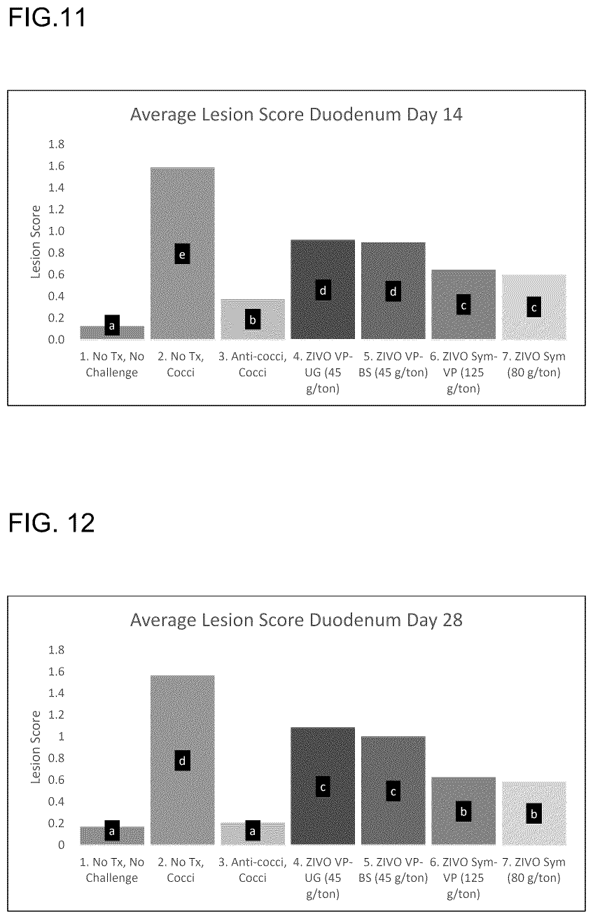

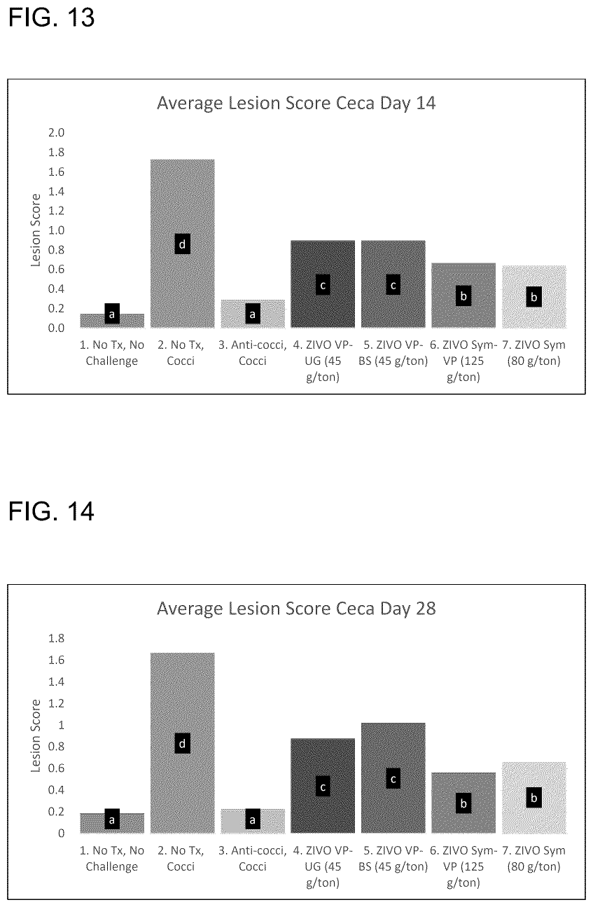

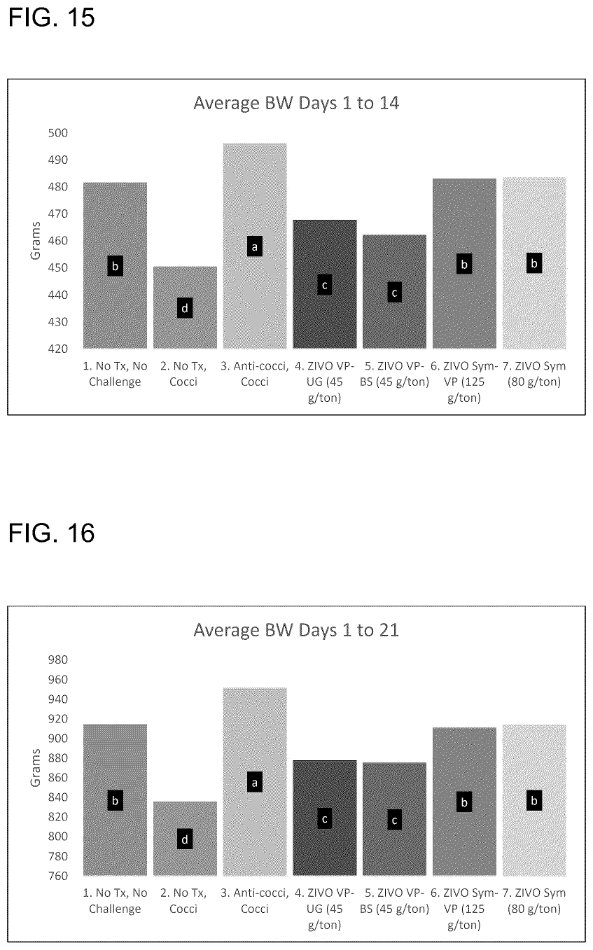

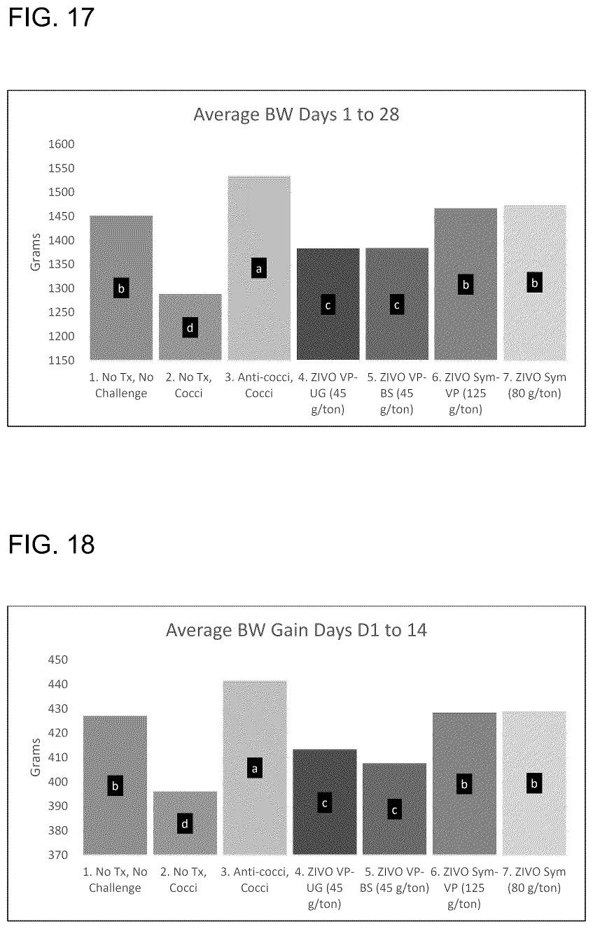

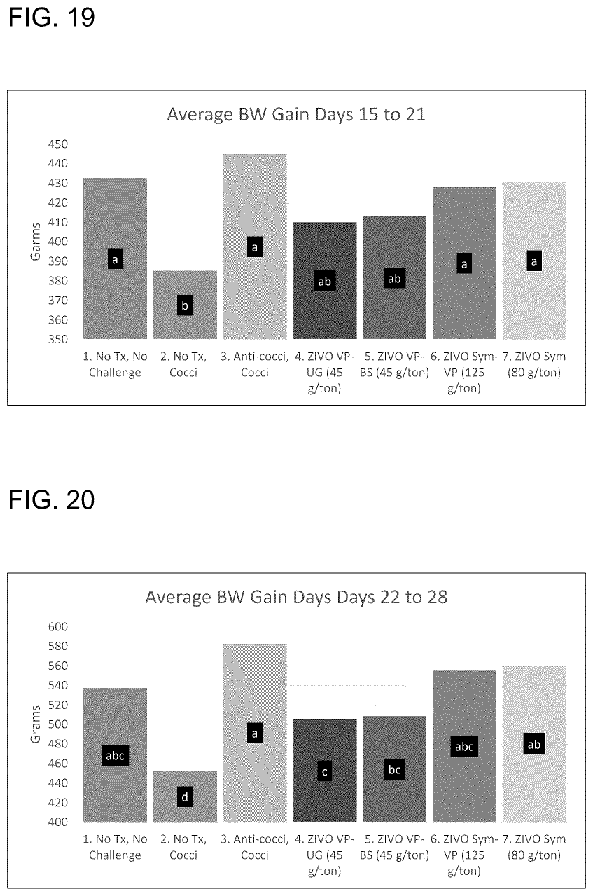

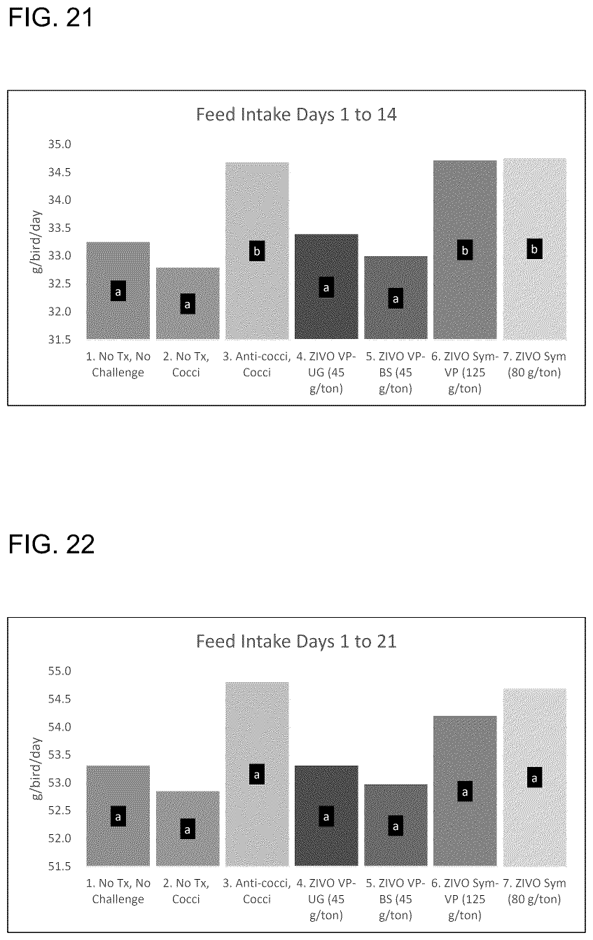

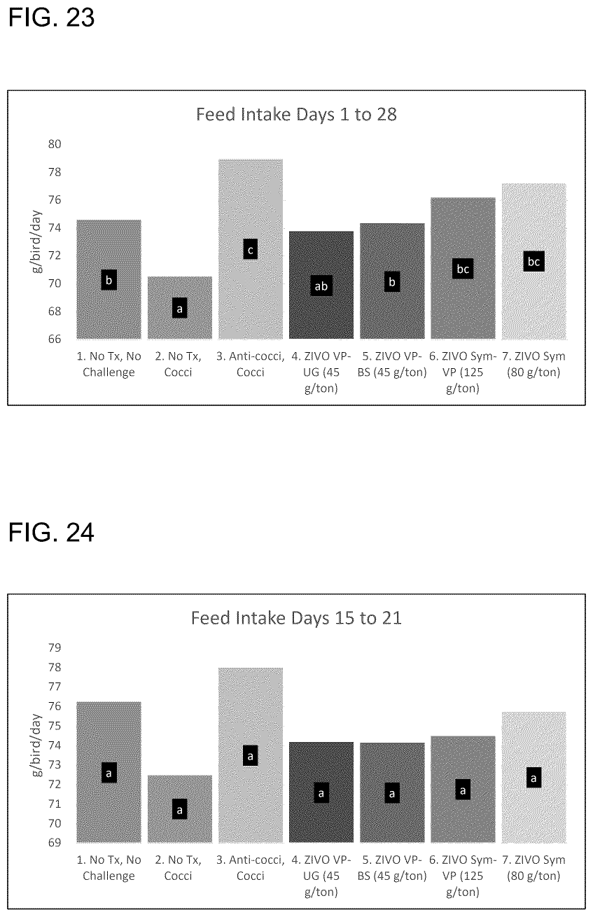

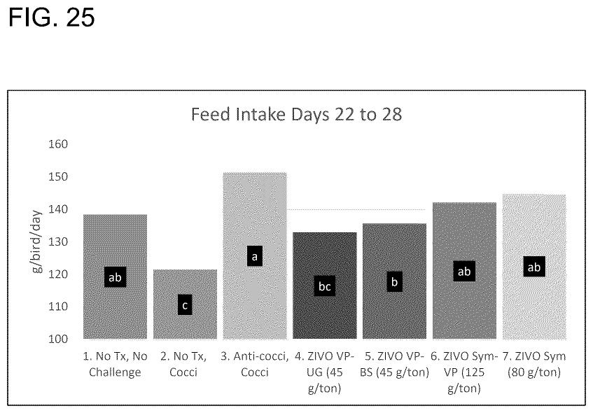

In general, analysis of the results based on the above tables and the accompanying graphs illustrated in FIGS. 1-25 supports the conclusion that use of the innovative compound in the treatment of coccidiosis-challenged poultry demonstrates a significant improvement in the health of diseased poultry when compared with untreated poultry. The positive results noted below were identified in the different bacterial variations of the composition of the disclosed inventive concept.

The results are summarized as follows:

FCR showed improvement in the sample poultry treated with the disclosed composition compared with untreated disease-challenged birds.

Upon examination of sacrificed sample birds, it was found that the average lesion scores of both the duodenum and the ceca of sample poultry treated with the disclosed composition were lower than the scores of sacrificed untreated disease-challenged birds. In addition, mortality rates of sample poultry treated with the disclosed composition were lower than the mortality rates of untreated disease-challenged birds. The data demonstrate that when two or more of Algoriphagus sp., Bosea sp., Brevundimonas sp., Desulfovibrio sp., Microbacterium sp., Sphingomonas sp., and Variovorax sp. are included in the treatment compound improvement is seen in FCR, mortality, and lesion scores compared with the untreated disease-challenged birds.

Various combinations of bacteria produced different results. As a non-limiting example, and with respect to lesion scores, the study revealed that the combination of Bosnea nasdae, Microbacterium, and Sphingomonas in the treatment compound was not enhanced by the addition of Variovorax paradoxus. It was also shown that other combinations including Variovorax paradoxus demonstrated superior results.

Average body weight of sample poultry treated with the disclosed composition as greater than the average body weight of untreated disease-challenged birds.

The improvement of the overall health of disease-challenged poultry as a result of treatment with the disclosed inventive composition was achieved without the use of antibiotics.

Overall the inventive composition demonstrates a cost-effective and practical approach to the treatment of disease states in animals.

1-25. (canceled)

26. A composition for use in feed for the treatment of an enteric disease in poultry through modulation of at least one Toll-Like Receptor (TLR) pathway, the composition derived from the group consisting essentially of a biomass containing Gram-negative bacteria, a supernatant from an algal culture containing an algal organism and Gram-negative bacteria, a bacterial biomass containing Gram-negative bacteria, a lipopolysaccharide-Lipid A compound derived from a bacterial strain, a cellular component, fraction or extract derived from a bacterial strain containing lipopolysaccharide-Lipid A, and a lipopolysaccharide-Lipid A derivative from a bacterial strain, the composition including a bacterial biomass as a portion of finished feed, wherein the bacterial biomass includes three bacteria, Brevundimonas sp., Sphingomonas sp., and Microbacterium sp, and wherein the animals being treated are poultry.

27. The composition for use of claim 26, wherein the bacterial biomass is fed to the animal in an amount from about 60.0 g per ton of finished feed to about 150.0 g per ton of finished feed,
preferably the bacterial biomass is fed to the animal in an amount from about 100.0 g per ton of finished feed to about 150.0 g per ton of finished feed,
more preferably the bacterial biomass is fed to the animal in an amount from about 60.0 g per ton of finished feed to about 100.0 g per ton of finished feed.

28. The composition for use of claim 26, in which Brevundimonas sp represent between about 40.0 g to about 50.0 g per ton of finished feed, Sphingomonas sp. represent between about 25.0 g to about 35.0 g per ton of finished feed, and Microbacterium sp represent between about 1.0 g and 10.0 g per ton of finished feed.

29. The composition for use of claim 26, wherein the bacterial biomass preferably includes Variovorax sp., Brevundimonas sp., Sphingomonas sp., and Microbacterium sp.,
and more preferably Variovorax sp, and Brevundimonas sp represent between about 40.0 g to about 50.0 g per ton of finished feed, Sphingomonas sp represent between about 25.0 g to about 35.0 g per ton of finished feed, and Microbacterium sp represent between about 1.0 g and 10.0 g per ton of finished feed.

30. A biomass-based composition for use in poultry feed derived from the group consisting essentially of a biomass containing Gram-negative bacteria, a supernatant from an algal culture containing an algal organism and Gram-negative bacteria, a bacterial biomass containing Gram-negative bacteria, a lipopolysaccharide-Lipid A compound derived from a bacterial strain, a cellular component, fraction or extract derived from a bacterial strain containing lipopolysaccharide-Lipid A, and a lipopolysaccharide-Lipid A derivative from a bacterial strain, more particularly the composition consisting essentially of three bacteria in the poultry feed in an amount from about 60.0 g per ton of finished feed to about 100.0 g per ton finished feed,
wherein the three bacteria include Brevundimonas sp., Sphingomonas sp., and Microbacterium sp. at amounts efficacious for the treatment of the subclinical or clinical coccidiosis, for use in a method for treating the animal for subclinical or clinical coccidiosis through modulation of at least one Toll-Like Receptor (TLR) pathway, comprising the step of administering the composition,
or
wherein the biomass-based composition consists essentially of four bacteria included in the poultry feed in an amount from about 100.0 g per ton of finished feed to about 150.0 g per ton of finished feed,
wherein the four bacteria include Variovorax sp., Brevundimonas sp., Sphingomonas sp., and Microbacterium sp.
